(19) United States
(12) Patent Application Publication
(10) Pub. No.: US 2020/0305828 A1
(43) Pub. Date: Oct. 1, 2020

(54) Radiation Image Processing Apparatus and Radiation Image Processing Method

(71) Applicant: Shimadzu Corporation, Kyoto (JP)

(72) Inventors: Takanori YOSHIDA, Kyoto (JP); Keiichi GOTO, Kyoto (JP)

(21) Appl. No.: 16/366,824

(22) Filed: Mar. 27, 2019

(51) Int. Cl. A61B 6/00; A61B 6/12

(52) U.S. Cl. CPC A61B 6/5235; A61B 6/504; A61B 6/5288; A61B 6/487; A61B 6/481; A61B 6/12

(57) ABSTRACT

A radiation image processing apparatus includes an image processing unit. The image processing unit is configured to create a device map superimposition image by superimposing a device fixed image on a map image.

### CROSS-REFERENCE TO RELATED APPLICATIONS

The related application number JP2016-183052, entitled “Radiation image processing apparatus and radiation image processing method”, filed on Sep. 20, 2016, invented by Takanori Yoshida, and Keiichi Goto, upon which this patent application is based is hereby incorporated by reference.

### BACKGROUND OF THE INVENTION

### Field of the Invention

The present invention relates to a radiation image processing apparatus and a radiation image processing method.

### Background Art

In the related art, there is a radiation image processing apparatus which processes a fluoroscopic image obtained by imaging a medical device introduced into the body of a subject (patient). Such a radiation image processing apparatus is disclosed in, for example, JP-T-2006-506117.

JP-T-2006-506117 discloses an image processing apparatus which processes an X-ray image (fluoroscopic image) of a stent (device) of which a catheter is introduced into the body of a subject (patient) in intravascular intervention treatment. The stent or a blood vessel portion into which the stent is inserted has a low visibility in the fluoroscopic image. In the image processing apparatus, a motion vector of a guide wire tip end for carrying the stent is generated, respective fluoroscopic images are positioned with each other by using the motion vector such that guide wire tip ends of the sequentially acquired fluoroscopic images match each other, the respective positioned fluoroscopic images are temporally integrated, and thus the stent and a blood vessel inner wall where the stent is disposed are emphasized.

However, in the emphasis process using temporal integration of images, it cannot be said that the visibility of the blood vessel inner wall which is scarcely reflected in a fluoroscopic image is sufficiently improved. In a case where a contrast agent is used, the visibility of a blood vessel portion is considerably improved, but the device (stent) present inside a blood vessel is buried with the contrast agent, and thus the visibility thereof is reduced. For example, in order to understand a state in which the stent is placed and thus to determine the necessity for additional blood vessel expansion or additional stent placement, it is highly necessary to check both of the stent and the blood vessel inner wall. Therefore, it is desirable to more clearly and simultaneously check both of a device introduced into a subject and a blood vessel portion in a fluoroscopic image.

The present invention has been made in order to solve the problem, and an object of the present invention is to provide a radiation image processing apparatus and a radiation image processing method capable of clearly and simultaneously check both of a device introduced into a subject and a blood vessel portion in a fluoroscopic image.

### SUMMARY OF THE INVENTION

In order to achieve the object, according to a first aspect of the present invention, there is provided a radiation image processing apparatus including an image generation unit that generates a fluoroscopic image based on a detection signal for radiation which is transmitted through a subject; a storage section that stores the fluoroscopic image generated by the image generation unit; and an image processing unit that performs image processing on the fluoroscopic image generated by the image generation unit, in which the image processing unit is configured to create a device fixed image which is positioned such that positions of a device introduced into a subject match each other in a plurality of the consecutively generated fluoroscopic images, create a map image in which a blood vessel portion is displayed in an identifiable manner with respect to the device by using a contrast image of a blood vessel of the subject, stored in the storage section, and create a device map superimposition image by superimposing the device fixed image on the map image.

In the radiation image processing apparatus according to the first aspect of the present invention, as described above, the image processing unit is configured to create a device fixed image which is positioned such that positions of a device introduced into the subject match each other in a plurality of the consecutively generated fluoroscopic images, create a map image in which a blood vessel portion is displayed in an identifiable manner with respect to the device by using a contrast image of a blood vessel of the subject, stored in the storage section, and create a device map superimposition image by superimposing the device fixed image on the map image. Consequently, in the map image, the blood vessel portion can be clearly visually recognized by using the contrast image, and the blood vessel portion can be displayed such that the device is identifiable. In the device map superimposition image, the device of the device fixed image is superimposed on the blood vessel portion of the map image, and thus the device buried with a contrast agent in a typical contrast image and the clear blood vessel portion obtained from the contrast image can be displayed together. As a result, both of the device introduced into the subject and the blood vessel portion can be clearly checked in the fluoroscopic image together.

In the radiation image processing apparatus according to the first aspect, preferably, the image processing unit is configured to perform a process of inverting a pixel value of the blood vessel portion in the contrast image or a process of changing a color of the blood vessel portion in the contrast image to a color which is identifiable with respect to the device, so as to generate the map image. Here, the reason why the device is buried during contrasting is that the blood vessel portion becomes black (a pixel value thereof become small) equivalent to or more than the device as a result of a contrast agent absorbing radiation. Thus, a pixel value of the blood vessel portion in the contrast image is inverted, or the blood vessel portion is displayed in other colors such that the device is visually recognizable, and thus it is possible to easily generate the map image in which the identification of the device can be improved. As a result, both of the device and the blood vessel portion are can be more clearly checked in the device map superimposition image.

In this case, preferably, the image processing unit is configured to extract a contour of the blood vessel portion in the contrast image so as to generate the map image in which the contour of the blood vessel portion is displayed in an identifiable manner. With this configuration, in the device map superimposition image, only the contour (that is, a blood vessel wall) of the blood vessel portion is displayed in an identifiable manner, and the device fixed image can be displayed inside the blood vessel portion. Consequently, it is possible to generate the device map superimposition image which does not give discomfort to a user familiar to the fluoroscopic image unlike a case where the blood vessel portion is entirely painted in an identifiable display color and in which both of the device and the blood vessel portion are clearly checked.

In the radiation image processing apparatus according to the first aspect, preferably, the image processing unit is configured to remove a part or the whole of a background portion other than the blood vessel portion in the contrast image by using a plurality of the consecutively generated contrast images, so as to create the map image. With this configuration, since the background portion other than the blood vessel portion can be removed from the map image, multiplexing of the background portion in the device map superimposition image can be suppressed, and thus the visibility of the entire image can be improved.

In this case, preferably, the image processing unit is configured to create a difference image between the contrast image and a past contrast image of the previous frame of the contrast image, and perform a threshold value process of removing an image component by using a threshold value of a pixel value on the difference image, so as to remove the background portion. With this configuration, in a case of a contrast image during cardiovascular intervention treatment, it is possible to easily remove the background portion while leaving the blood vessel portion by using the fact that a blood vessel position changes in consecutive frames but a position of a bone or an organ which is scarcely moved does not change.

In the radiation image processing apparatus according to the first aspect, preferably, the image processing unit is configured to acquire heartbeat phase information of the fluoroscopic image from an electrocardiographic waveform or the fluoroscopic image, select the contrast image acquired in a heartbeat phase which substantially matches a heartbeat phase of the device fixed image from among a plurality of the contrast images stored in the storage section on the basis of the heartbeat phase information, and create the map image by using the selected contrast image. With this configuration, in a case of a contrast image during cardiovascular intervention treatment, since movement of the blood vessel portion is periodic motion caused by beating of the heart, the map image which accurately matches the device fixed image can be selected on the basis of matching of a heartbeat phase.

In the radiation image processing apparatus according to the first aspect, preferably, the image processing unit is configured to create a partial map image in the vicinity of the device in the contrast image generated during contrasting, and superimpose the device fixed image generated after finishing of the contrasting on the partial map image on the basis of a position of the device, so as to create the device map superimposition image. With this configuration, the device map superimposition image can be created immediately after contrasting is finished on the basis of a device position by using a series of fluoroscopic images (contrast images) acquired during contrasting and after finishing of contrasting.

In the radiation image processing apparatus according to the first aspect, preferably, the image processing unit is configured to create the device fixed image on the basis of a device emphasis image in which the device is emphasized by using the fluoroscopic images of a plurality of consecutive frames. With this configuration, it is possible to improve the visibility of the device in the device fixed image. As a result, both of the device and the blood vessel portion are can be more clearly checked in the device map superimposition image.

In the radiation image processing apparatus according to the first aspect, preferably, the device includes a stent for blood vessel treatment, and the fluoroscopic image and the contrast image are radiation images of a part which is periodically moved due to a heartbeat of the subject. In a case where body tissue including a blood vessel is moved periodically due to a heartbeat, such as cardiovascular intervention treatment, it is hard to sufficiently improve visibility of each of the stent and the blood vessel portion. Therefore, the present invention in which the blood vessel portion and the device are identifiable by using the map image while suppressing a position change by using the device fixed image is considerably useful in this case.

According to a second aspect of the present invention, there is provided a radiation image processing method including acquiring a contrast image of a blood vessel of a subject through radiation fluoroscopic imaging; consecutively acquiring fluoroscopic images of the subject through radiation fluoroscopic imaging; creating a device fixed image which is positioned such that positions of a device introduced into the subject match each other in a plurality of the consecutively generated fluoroscopic images; creating a map image in which a blood vessel portion is displayed in an identifiable manner with respect to the device by using the contrast image; and creating a device map superimposition image by superimposing the device fixed image on the map image.

The radiation image processing method according to the second aspect of the present invention includes creating a device fixed image which is positioned such that positions of a device introduced into the subject match each other in a plurality of the consecutively generated fluoroscopic images, creating a map image in which a blood vessel portion is displayed in an identifiable manner with respect to the device by using the contrast image, and creating a device map superimposition image by superimposing the device fixed image on the map image. Consequently, in the map image, the blood vessel portion can be clearly visually recognized by using the contrast image, and the blood vessel portion can be displayed such that the device is identifiable. In the device map superimposition image, the device of the device fixed image is superimposed on the blood vessel portion of the map image, and thus the device buried with a contrast agent in a typical contrast image and the clear blood vessel portion obtained from the contrast image can be displayed together. As a result, both of the device introduced into the subject and the blood vessel portion can be clearly checked in the fluoroscopic image together.

### BRIEF DESCRIPTION OF THE DRAWINGS

FIG. 1 is a block diagram illustrating the overall configuration of a radiation imaging apparatus including an image processing apparatus according to a first embodiment of the present invention.

FIG. 2A is a diagram illustrating an example of a device.

FIG. 2B is a diagram for explaining a state of the device in a blood vessel.

FIG. 2C is a diagram for explaining a state of the device in a blood vessel.

FIG. 3 is a diagram for explaining image processing performed by the image processing apparatus.

FIG. 4 is a diagram for explaining a process of creating a device fixed image.

FIG. 5 is a diagram for explaining a process of removing a background portion.

FIG. 6 is a diagram for explaining a process of creating a device emphasis image.

FIG. 7 is a flowchart for explaining a flow of image processing performed by the image processing apparatus.

FIG. 8 is a diagram for explaining image processing performed by an image processing apparatus according to a second embodiment.

FIG. 9 is a diagram for explaining a process of automatically selecting a contrast image in an image processing apparatus according to a third embodiment.

FIG. 10 is a diagram for explaining a process of creating a partial map image in an image processing apparatus according to a fourth embodiment.

### DETAILED DESCRIPTION OF THE INVENTION

Hereinafter, embodiments of the present invention will be described with reference to the drawings.

### First Embodiment

Configuration of Radiation Image Processing Apparatus

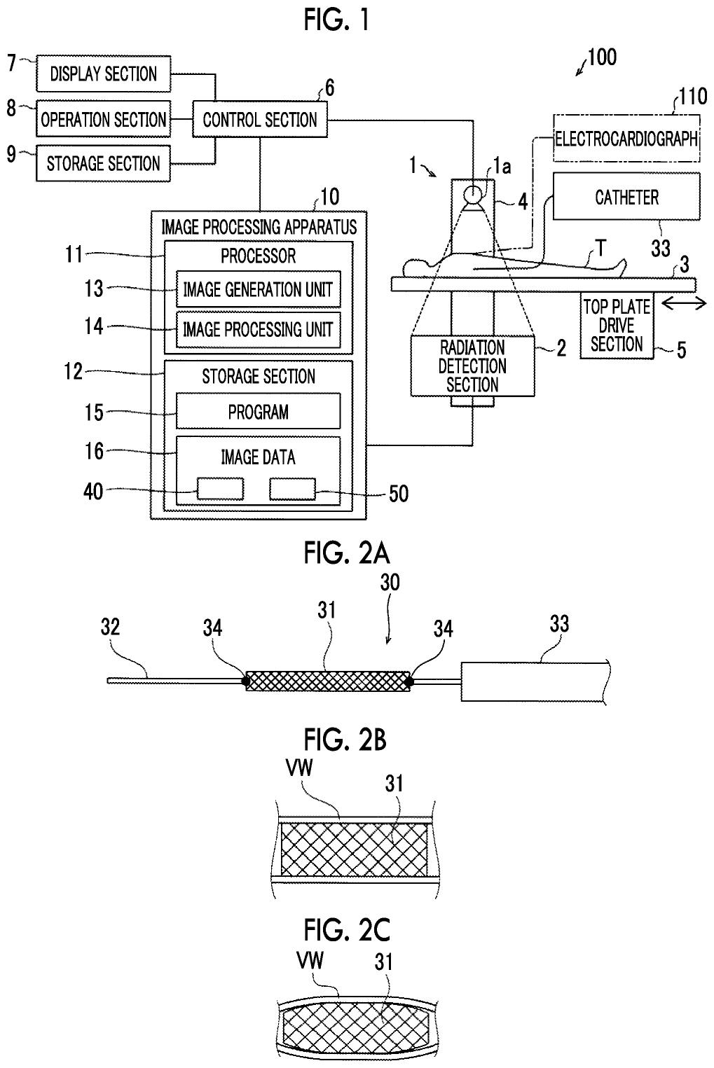

With reference to FIGS. 1 to 6, a description will be made of a configuration of an image processing apparatus according to a first embodiment of the present invention. The image processing apparatus 10 is an example of a “radiation image processing apparatus” in the claims.

The image processing apparatus 10 according to the first embodiment is configured to perform image processing in real time during capturing of a fluoroscopic image in combination with a radiation imaging apparatus 100 which captures a radiation image. The radiation imaging apparatus 100 is an apparatus which applies radiation from the outside of a subject T such as a human body, and thus captures a radiation image (fluoroscopic image) obtained by imaging the inside of the subject T. The radiation imaging apparatus 100 is an X-ray imaging apparatus which captures an X-ray image by using an X-ray which is an example of radiation.

The radiation imaging apparatus 100 includes an irradiation section 1 which irradiates the subject T with radiation (X-ray) and a radiation detection section 2 which detects the radiation transmitted through the subject T. The irradiation section 1 and the radiation detection section 2 are disposed to be opposed to each other with a top plate 3, interposed therebetween, on which the subject T is mounted. The irradiation section 1 and the radiation detection section 2 are movably supported by a movement mechanism 4. The top plate 3 is movable in a horizontal direction by a top plate drive section 5. The irradiation section 1, the radiation detection section 2, and the top plate 3 are moved via the movement mechanism 4 and the top plate drive section 5 such that a region of interest in the subject T is imaged. The region of interest is an imaging target region in the subject T in order to perform an examination or treatment. The radiation imaging apparatus 100 includes a control section 6 which controls the movement mechanism 4 and the top plate drive section 5.

The irradiation section 1 includes a radiation source 1a. The radiation source 1a is an X-ray tube which is connected to a high voltage generation section (not illustrated), and generates an X-ray as a result of a high voltage being applied thereto. The radiation source 1a is disposed in a state in which an X-ray emission direction is directed to a detection surface of the radiation detection section 2. The irradiation section 1 is connected to the control section 6. The control section 6 controls the irradiation section 1 according to preset imaging conditions such as a tube voltage, a tube current, and a time interval of X-ray irradiation, so as to generate an X-ray from the radiation source 1a.

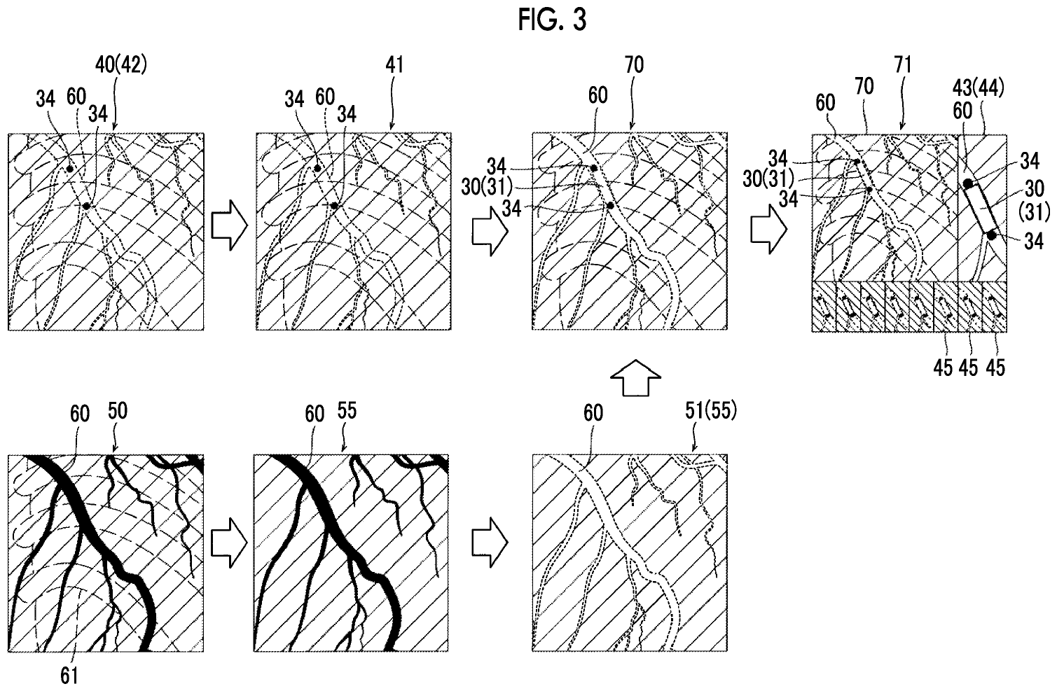

The radiation detection section 2 detects an X-ray which is applied from the irradiation section 1 and is transmitted through the subject T, and outputs a detection signal corresponding to a detected X-ray intensity. The radiation detection section 2 is configured with, for example, a flat panel detector (FPD). The radiation detection section 2 outputs an X-ray detection signal with a predetermined resolution to the image processing apparatus 10. The image processing apparatus 10 acquires the X-ray detection signal from the radiation detection section 2, and generates a fluoroscopic image 40 (refer to FIG. 3).

The control section 6 is a computer configured to include a central processing unit (CPU), a read only memory (ROM), and a random access memory (RAM). The control section 6 controls each section of the radiation imaging apparatus 100 by the CPU executing a predetermined control program. The control section 6 performs control of the irradiation section 1 and the image processing apparatus 10 or drive control of the movement mechanism 4 and the top plate drive section 5.

The radiation imaging apparatus 100 includes a display section 7, an operation section 8, and a storage section 9. The display section 7 is a monitor such as a liquid crystal display. The operation section 8 is configured to include, for example, a keyboard, a mouse, and a touch panel, or other controllers. The storage section 9 is configured with a storage device such as a hard disk drive. The control section 6 is configured to perform control of displaying an image generated by the image processing apparatus 10 on the display section 7. The control section 6 is configured to receive an input operation using the operation section 8. The storage section 9 is configured to store image data, imaging conditions, and various set values. Each of the display section 7 and the operation section 8 may be provided in the image processing apparatus 10.

The image processing apparatus 10 is a computer configured to include a processor 11 such as a CPU or a graphics processing unit (GPU), and a storage section 12 such as a ROM and a RAM. In other words, the image processing apparatus 10 is configured to cause the processor 11 to execute an image processing program stored in the storage section 12. The image processing apparatus 10 may be integrally configured with the control section 6 by causing the same hardware (CPU) as the control section 6 to execute the image processing program.

The storage section 12 stores a program 15 (image processing program) for causing a computer to function as the image processing apparatus 10. In the first embodiment, the storage section 12 is configured to store image data 16 including the fluoroscopic image 40 and a contrast image 50 which will be described later, which are generated by an image generation unit 13 which will be described later.

The image processing apparatus 10 includes the image generation unit 13 and an image processing unit 14 as functions realized by executing the image processing program 15. The image generation unit 13 and the image processing unit 14 may be configured separately from each other by dedicated processors.

The image generation unit 13 is configured to generate the fluoroscopic image 40 based on a detection signal of radiation transmitted through the subject T. The image generation unit 13 consecutively generates the fluoroscopic images 40 in a moving image form on the basis of detection signals from the radiation detection section 2. In other words, X-rays are intermittently applied to the subject T from the irradiation section 1 at a predetermined time interval, and X-rays transmitted through the subject T are sequentially detected by the radiation detection section 2. The image generation unit 13 images detection signals which are sequentially output from the radiation detection section 2, and thus consecutively generates the fluoroscopic images 40 at a predetermined frame rate. The frame rate is, for example, about 15 FPS to 30 FPS. The fluoroscopic image 40 is, for example, an image having a pixel value of a predetermined grayscale number (for example, 10 to 12 bits) in terms of grayscale. Thus, the fluoroscopic image is displayed black (dark) in a pixel with a low pixel value, and is displayed white (bright) in a pixel with a high pixel value.

The image processing unit 14 is configured to perform image processing on the fluoroscopic image 40 generated by the image generation unit 13. Details of the image processing will be described later.

In the first embodiment, the image processing apparatus 10 (radiation imaging apparatus 100) is configured to generate the fluoroscopic image 40 of a device 30 (refer to FIG. 2A) introduced into the subject T and a contrast image 50 of a blood vessel of the subject T. In the first embodiment, the fluoroscopic image 40 and the contrast image 50 are radiation images of parts which are periodically moved due to heartbeats of the subject.

In the first embodiment, as illustrated in FIG. 2A, the device 30 introduced into the subject T includes a stent 31 for blood vessel treatment. The stent 31 is used for, for example, coronary artery (cardiovascular) intervention treatment. In the coronary artery intervention treatment, treatment is performed by inserting a catheter 33 having a guide wire 32 therein into a blood vessel of the subject T, and causing the catheter 33 to reach a coronary artery of the heart via the blood vessel. The stent 31 has a tubular shape having a mesh structure made of thin metal or the like. The stent 31 is disposed in a stenosed part of a blood vessel, and is placed in the blood vessel as a result of being expanded by using a balloon from the inside, so as to support the stenosed blood vessel from the inside. Therefore, the stent 31 having a mesh structure is hardly to be reflected in the fluoroscopic image 40, and thus markers 34 of which radiation transmission is low (or radiation transmission is zero) is provided at the stent 31, the balloon, or the like as marks. One or two markers 34 may be often provided.

In the coronary artery intervention treatment, a doctor sends the catheter 33 to a coronary artery of the heart while referring to the fluoroscopic images 40 which are moving images generated in real time by the image processing apparatus 10 (radiation imaging apparatus 100). During treatment, it is necessary to specify a stenosed part, to determine positions of the stent 31 and the balloon for blood vessel expansion in the stenosed part, and to check the stent 31 after being placed. Since blood in a blood vessel and peripheral body tissue have a small difference in X-ray transmission, and thus a blood vessel portion has low visibility in the fluoroscopic image 40. Therefore, before treatment is started or during treatment, the contrast image 50 (refer to FIG. 3) obtained by using a contrast agent is captured. A contrast agent has low radiation transmission in the same manner as the markers 34, and is thus reflected as a dark part (black part) in the fluoroscopic image 40. The contrast agent is injected into a blood vessel via the catheter, and thus a blood vessel portion 60 (refer to FIG. 3) which is scarcely reflected in the typical fluoroscopic image 40 can be clearly reflected in the contrast image 50.

As illustrated in FIGS. 2B and 2C, checking after the stent 31 is placed includes checking whether or not the stent 31 (a stenosed part of the blood vessel) is sufficiently expanded and checking whether or not the stent 31 is brought into close contact with a blood vessel wall VW. For example, in FIG. 2B, the stent 31 and the stenosed part are sufficiently expanded, and thus the stent 31 is brought into close contact with the blood vessel wall VW. In FIG. 2C, the stent 31 is not sufficiently expanded around end parts thereof, and close contact between the blood vessel wall VW and the stent 31 is not sufficient. A checking result is a basis of determination of whether or not additional expansion using a balloon is to be performed or an additional stent is to be placed.

Thus, in checking after the stent 31 is placed, it is important to check (visually recognize) both of the stent 31 and the blood vessel portion 60. However, as illustrated in FIG. 3, in the typical fluoroscopic image 40, a position of the stent 31 can be understood on the basis of the markers 34 reflected as black dots in the image, but the blood vessel portion 60 (refer to a dashed portion) is hardly visually recognized. In the contrast image 50, the blood vessel portion 60 into which the contrast agent is injected is clearly reflected as a black region, but the markers 34 (stent 31) in the blood vessel is buried with the contrast agent and is thus hardly visually recognized.

Therefore, in the first embodiment, as illustrated in FIG. 3, the image processing unit 14 is configured to perform image combination (superimposition) by using the fluoroscopic image 40 in which the device 30 (the stent 31 and the markers 34) is reflected to be visually recognized and the contrast image 50 in which the blood vessel portion 60 is reflected, and thus to create a device map superimposition image 70 in which both of the device 30 (the stent 31 and the markers 34) and the blood vessel portion 60 can be visually recognized.

Image Processing on Fluoroscopic Image

In the first embodiment, the image processing unit 14 is configured to perform a process of creating a device fixed image 41 in real time from a plurality of consecutively generated fluoroscopic images 40, a process of creating a map image 51 of the blood vessel portion 60 by using the contrast image 50, and a process of creating the device map superimposition image 70 by using the device fixed image 41 and the map image 51. Hereinafter, each of the processes will be described in detail. In the following description, for convenience of differentiation, a fluoroscopic image during contrasting will be referred to as the contrast image 50, and a fluoroscopic image during non-contrasting will be referred to as the fluoroscopic image 40.

Device Fixed Image

The device fixed image 41 is a fluoroscopic image which is positioned such that positions of the device 30 introduced into the subject T match each other in a plurality of consecutively generated fluoroscopic images 40. In the fluoroscopic images 40 which are generated as moving images in real time, the blood vessel portion 60 and the device 30 in the blood vessel portion 60 are normally periodically moved due to a heartbeat or breathing of the subject T. The device fixed image 41 displays a position of the device 30 reflected in the fluoroscopic image 40 in a fixed manner in the image (in a display screen).

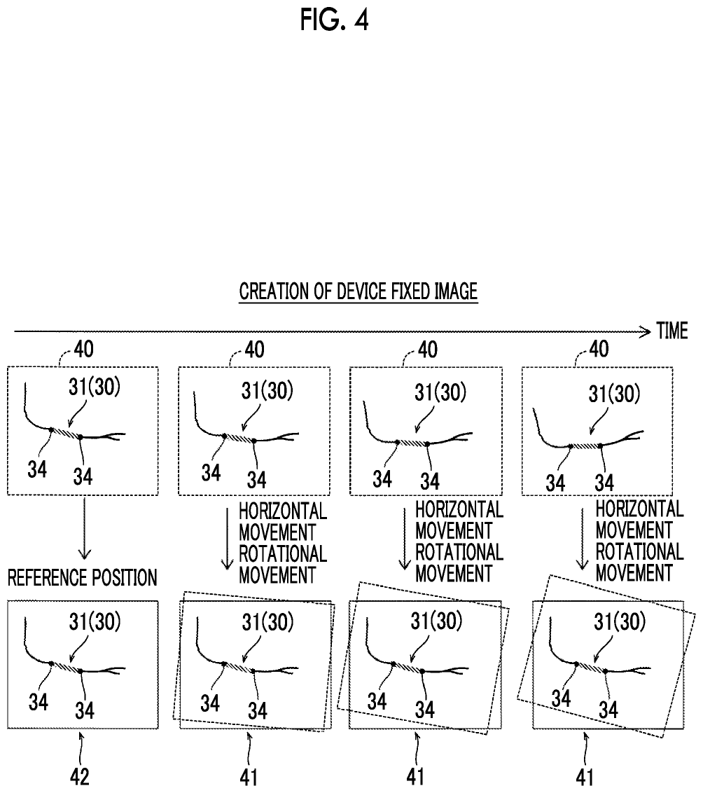

Specifically, as illustrated in FIG. 4, the image processing unit 14 detects the markers 34 of the device 30 from each of the fluoroscopic images 40 (non-contrast image) which are generated in real time by the image generation unit 13. The markers 34 may be detected by using a well-known image recognition technique. The image processing unit 14 acquires position coordinates of the markers 34 in the fluoroscopic image 40.

The image processing unit 14 selects a reference image 42 used as a reference of the device fixed image 41 at a predetermined timing from among the plurality of fluoroscopic images 40 generated as moving images. In other words, the image processing unit 14 selects an image of one frame from among the fluoroscopic images 40 as the reference image 42.

The image processing unit 14 positions the fluoroscopic image 40 of each frame after the reference image 42 such that positions of the respective markers 34 (device 30) match the positions of the markers 34 (device 30) reflected in the reference image 42. In other words, one or both of horizontal movement and rotational movement are performed on the fluoroscopic image 40 of each frame. Consequently, the device fixed image 41 in which positions of the markers 34 (device 30) match the positions of the markers 34 (device 30) reflected in the reference image 42 is created for each frame. As a result, a fluoroscopic image (device fixed image 41) in a state in which positions of the markers 34 (device 30) are fixed is consecutively output in frames after the reference image 42.

Map Image

As illustrated in FIG. 3, the map image 51 is an image which is created on the basis of the contrast image 50 of a blood vessel of the subject T, and displays the blood vessel portion 60 in an identifiable manner with respect to the device 30. In the first embodiment, before the device map superimposition image 70 is created, the contrast image 50 captured by injecting a contrast agent into a blood vessel is acquired in advance, and is stored in the storage section 12. The image processing unit 14 creates the map image 51 on the basis of the contrast image 50 of the blood vessel of the subject T, stored in the storage section 12.

The contrast image 50 is preferably stored in the storage section 12 for at least one cycle of the contrast image 50 which is periodically moved due to a heartbeat or breathing of the subject T. The image processing unit 14 selects the contrast image 50 to be superimposed on the device fixed image 41 from among a plurality of contrast images 50 corresponding to a predetermined time, stored in the storage section 12. In other words, the image processing unit 14 selects the contrast image 50 of which the blood vessel portion 60 matches or is approximate to the blood vessel portion 60 (refer to a dash line portion) of the device fixed image 41. In the device fixed image 41 and the contrast image 50, the blood vessel portions 60 are not required to entirely match each other or to be entirely approximate to each other. Since the device fixed image 41 is a moving image in which a position of the device 30 (markers 34) is fixed, and parts other than the device 30 (markers 34) are moved, in the contrast image 50, the blood vessel portion 60 in a peripheral region of a position (positions of the markers 34) of the device 30 may match that of the device fixed image 41.

The contrast image 50 may be automatically selected by the image processing unit 14, and may be manually selected by receiving a selection operation from a user (a doctor performing intervention treatment). The selection operation is performed, for example, by receiving an operation of selecting any one contrast image 50 via the operation section 8 from among a plurality of contrast images 50 which are displayed in a list form on the display section 7. An example in which the image processing unit 14 automatically selects the contrast image 50 will be described in a third embodiment which will be described later.

The image processing unit 14 creates the map image which displays the blood vessel portion 60 in an identifiable manner with respect to the device 30 by using the selected contrast image 50. Displaying the blood vessel portion 60 in an identifiable manner with respect to the device 30 indicates that an image portion (pixels) of the device 30 (stent 31) and an image portion (pixels) of the blood vessel portion 60 have light and shade (pixel value) or color contrast to the degree of being identifiable to a user.

In the first embodiment, the image processing unit 14 is configured to perform a process of inverting a pixel value of the blood vessel portion 60 in the contrast image 50 or a process of changing a color of the blood vessel portion 60 in the contrast image 50 to a color which is identifiable with respect to the device 30, so as to generate the map image 51. As illustrated in FIG. 3, the blood vessel portion 60 in the contrast image 50 has a small pixel value (in other words, displayed black) due to a contrast agent. The device 30 in the fluoroscopic image (device fixed image 41) also tends to have a small pixel value (that is, displayed black) compared with peripheral body tissue. A pixel value of the blood vessel portion 60 in the contrast image 50 is inverted, and thus an inversion image in which a pixel value of the blood vessel portion 60 is great (that is, the blood vessel portion 60 is displayed white). Consequently, in a case where the device fixed image 41 is superimposed on the map image 51 configured with the inversion image, the device and the markers 34 are reflected black to be identifiable in the blood vessel portion 60 with the white background obtained through pixel inversion.

There is no limitation to inversion of a pixel value, and the blood vessel portion 60 may be replaced with other colors which are different from black and white (grayscale). Instead of white (inverted pixel value) in an inversion image, for example, the device 30 and the blood vessel portion 60 can be displayed to be identified from each other by changing the black (gray) device 30 to a predetermined color such as yellow or green which can be differentiated from the color of the device 30.

Removal of Background Portion

In creation of the map image 51, the selected contrast image 50 may be used without being changed, but an image from which a background portion 61 is removed by using a plurality of consecutively generated contrast images 50 may be used. In the first embodiment, preferably, the image processing unit 14 is configured to remove a part or the whole of the background portion 61 other than the blood vessel portion 60 in the contrast image 50 by using a plurality of consecutively generated contrast images 50, so as to create the map image 51.

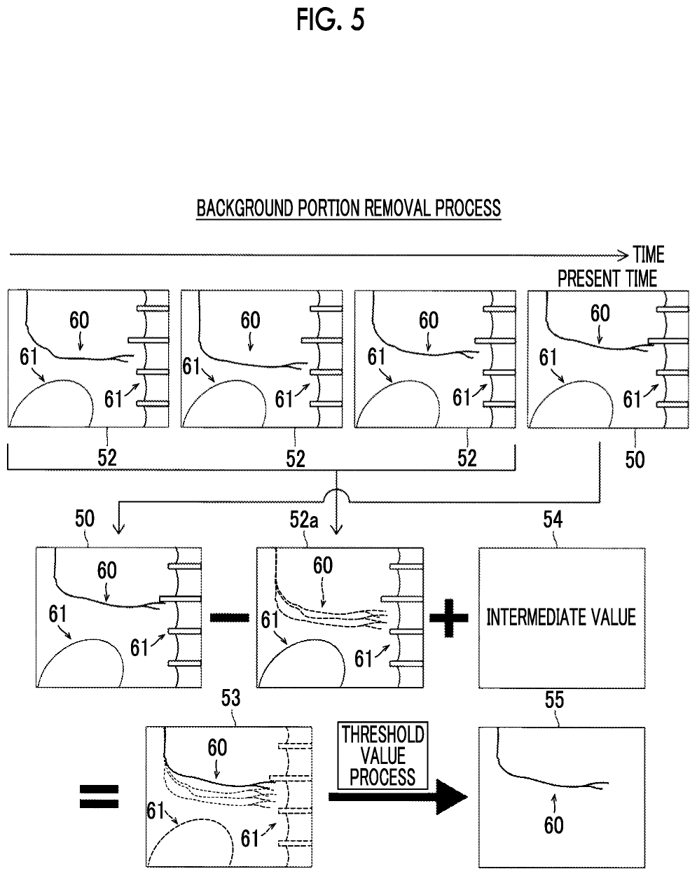

Specifically, as illustrated in FIG. 5, the image processing unit 14 creates a difference image 53 between the current contrast image 50 and a past contrast image 52 of the previous frame of the current contrast image 50. FIG. 5 illustrates an example in which past contrast images 52 of, for example, (previous) three frames are used. First, the image processing unit 14 creates a combined image 52a of a plurality of past contrast images 52. For example, the image processing unit 14 creates the combined image 52a (average image) in which respective pixel values of the plurality of past contrast images 52 are averaged. The blood vessel portion 60 or the like which changes due to a heart stroke is reflected to be shaken (blurred) in the combined image 52a, and has a color close to gray since pixel values are uniform through averaging. The background portion 61 of a bone or an organ which does not temporally change does not change even through averaging, and is thus reflected in the combined image 52a in the same manner as in the past contrast image 52.

The image processing unit 14 may create the combined image 52a in which a plurality of past contrast images 52 are added together at temporally different ratios. For example, the image processing unit 14 combines a plurality of past contrast images 52 by using a recursive filter which is a time filter. In this case, as the past contrast image 52 having a frame closer to a frame of the current contrast image 50, the past contrast image 52 is combined to occupy a higher ratio in the combined image 52a.

The image processing unit 14 performs a difference process (subtraction) between the current contrast image and the combined image 52a so as to create the difference image 53. The image processing unit 14 performs a calculation process (addition and subtraction processes on pixel values between corresponding pixels) of A−B+C on the current contrast image 50 (indicated by an image A), the combined image 52a (indicated by an image B), and an intermediate value image 54 (indicated by an image C) configured with intermediate values of pixel values, so as to create the difference image 53. The blood vessel portion 60 in the current contrast image 50 is reflected black (that is, has a small pixel value) in the difference image 53. In the difference image 53, the shaken blood vessel portion 60 in the combined image 52a is reflected whiter (that is, has a greater pixel value) and the background portion 61 which is not moved is reflected gray (near an intermediate value). In other words, in the difference image 53, contrast (pixel value difference) between the blood vessel portion 60 in the current contrast image 50 and the background portion 61 or the blood vessel portion 60 averaged in the combined image 52a becomes clear.

The image processing unit 14 performs a threshold value process of removing an image component by using a threshold value of a pixel value on the difference image 53, so as to remove the background portion 61. Since a pixel value difference between the blood vessel portion 60 in the current contrast image 50 and the background portion 61 or the like is increased in the difference image 53, a threshold value is set between the blood vessel portion 60 and the background portion 61 or the like, and thus the background portion 61 is easily removed. As a result, the image processing unit 14 acquires a background removed image 55 in which the blood vessel portion 60 is reflected, and the background portion 61 other than the blood vessel portion 60 is removed. The image processing unit 14 can create the map image 51 by using the background removed image 55.

Device Map Superimposition Image

As illustrated in FIG. 3, the image processing unit 14 is configured to superimpose the device fixed image 41 on the map image 51 so as to create the device map superimposition image 70. The image processing unit 14 creates the device fixed image 41 by using the fluoroscopic image 40 which is generated in real time, and sequentially superimposes the device fixed image 41 on the map image 51.

As described above, since the device fixed image 41 is positioned at a position of the device 30 (markers 34), the device 30 (markers 34) and the peripheral blood vessel portion 60 of the device 30 are displayed at fixed positions. In the selected contrast image 50, the map image 51 and the device fixed image 41 are superimposed on each other such that a position of the blood vessel portion 60 in the vicinity of the device 30 matches or is approximate to that in the device fixed image 41 (reference image 42), and thus the blood vessel portion 60 in the map image 51 is superimposed on the blood vessel portion 60 having a low visibility in the device fixed image 41. As a result, the device map superimposition image 70 is a fluoroscopic image in which the device 30 of the device fixed image 41 is disposed in the blood vessel portion 60 of the map image 51, and thus the identification of both of the blood vessel portion 60 and the device 30 is improved.

Device Emphasis Process

In the first embodiment, an emphasis process of displaying the device 30 in the device fixed image 41 in an emphasized manner may be performed.

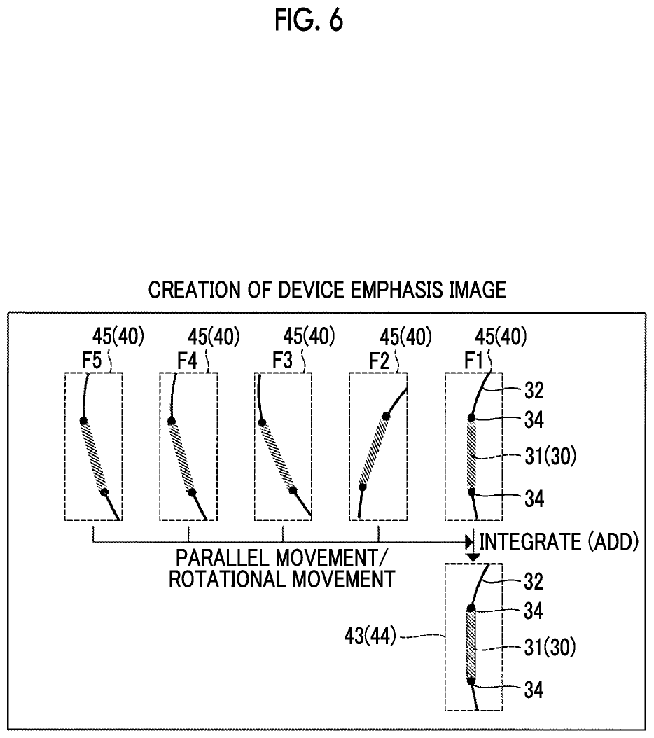

In other words, in the first embodiment, preferably, the image processing unit 14 is configured to create the device fixed image 41 on the basis of a device emphasis image 43 in which the device 30 is emphasized by using the fluoroscopic images 40 of a plurality of consecutive frames. Specifically, as illustrated in FIG. 6, the image processing unit 14 generates the device emphasis image 43 by positioning and superimposing the device 30 (markers 34) (that is, by performing an integration process on images) by using the fluoroscopic images 40 of a plurality of previous frames including the fluoroscopic image 40 of the latest frame F1. The number of superimposed images is any number, but FIG. 6 illustrates an example of images of five frames (that is, F1 to F5) including the latest frame F1.

The visibility of the device 30 is further improved by creating the device map superimposition image 70 by using the device emphasis image 43 as the device fixed image 41.

Device Enlargement Process

As illustrated in FIG. 3, in the first embodiment, an enlargement display process of displaying the enlarged device 30 in the device map superimposition image 70 may be performed.

In other words, in the first embodiment, the image processing unit 14 is configured to enlarge and cutout (trim) an image of the device 30 in the device map superimposition image 70 so as to display an enlarged image 44 of the device 30. The image processing apparatus 10 generates, for example, a display image 71 in which the fluoroscopic image 40 of the latest frame and the enlarged image 44 of the stent 31 are displayed side by side, and outputs the display image 71 to the control section 6 (display section 7). The device emphasis image 43 may be applied to only the enlarged image 44. The display image 71 in FIG. 3 illustrates an example of a form in which the device map superimposition image 70, the enlarged image 44, and a plurality of trimming images 45 (refer to FIGS. 3 and 6) used to apply an emphasis process to the enlarged image 44 are displayed side by side.

In the device map superimposition image 70, the background is moved in regions other than a peripheral region of the device 30 (markers 34) positioned in the device fixed image 41. On the other hand, in the enlarged image 44, only the peripheral region of the positioned device 30 (markers 34) is enlarged, so that the moved background portion is removed, and thus the enlarged image is substantially a still image. Thus, in a case of paying attention to the image, the visibility thereof is improved.

Process Operation of Image Processing Apparatus

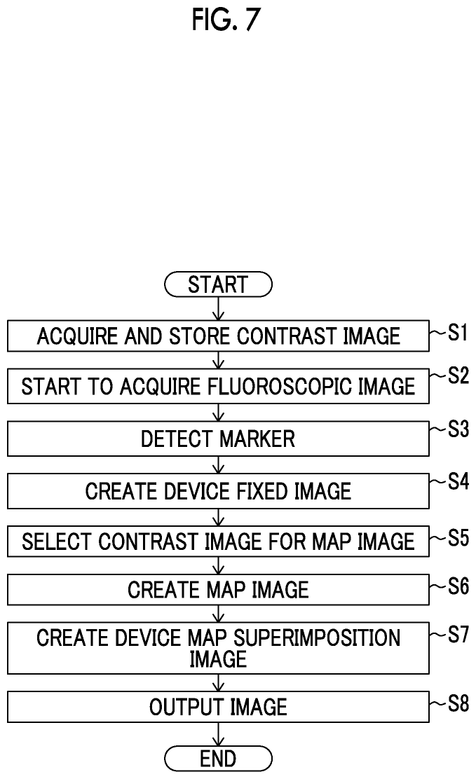

Next, with reference to FIG. 7, a description will be made of a process operation of the image processing apparatus 10.

In step S1 in FIG. 7, the image processing apparatus 10 acquires the contrast image 50, and stores the contrast image 50 in the storage section 12. In other words, the image processing apparatus 10 acquires a detection signal from the radiation detection section 2 which detects an X-ray which is applied from the irradiation section 1 and is transmitted through the subject T in a contrasting state in which a contrast agent is injected into a blood vessel. The image generation unit 13 generates the contrast image on the basis of the acquired detection signal. A plurality of contrast images 50 are generated for a period which is equal to or longer than one cycle of a heart stroke, and each thereof is stored in the storage section 12.

In step S2, the image processing apparatus 10 starts to acquire the fluoroscopic image 40. In other words, a detection signal is acquired from the radiation detection section 2 which detects an X-ray which is applied from the irradiation section 1 and is transmitted through the subject T in a non-contrasting state. The image generation unit 13 generates the fluoroscopic image 40 on the basis of the acquired detection signal. The fluoroscopic image 40 is consecutively generated in the frame unit as a moving image, and is output to the image processing unit 14.

In step S3, the image processing unit 14 detects the markers 34 through image recognition from the generated fluoroscopic image 40. The image processing unit 14 acquires positions of the markers 34 (that is, a position of the device 30) in the fluoroscopic image 40.

In step S4, the image processing unit 14 creates the device fixed image 41. In other words, the image processing unit 14 selects the reference image 42 from among the fluoroscopic images 40 of respective frames, and aligns each fluoroscopic image 40 after a frame of the reference image 42 with marker positions of the reference image 42, so as to create the device fixed image 41. In a case where the device emphasis image 43 is created, the image processing unit 14 integrates (adds) the fluoroscopic images 40 of a plurality of consecutive frames so as to create the device emphasis image 43 as illustrated in FIG. 6.

In step S5, the image processing unit 14 selects the contrast image 50 used to create the map image 51. The image processing unit 14 selects the contrast image 50 of which the blood vessel portion 60 matches or is approximate to the blood vessel portion 60 of the device fixed image 41. As described above, the map image 51 is manually selected by receiving a selection from a user (doctor) or is automatically selected by the image processing unit 14.

In step S6, the image processing unit 14 creates the map image 51 by using the selected contrast image 50. In a case where the background portion 61 is removed, the image processing unit 14 creates the difference image 53 by using the past contrast images 52 of a plurality of previous frames of the selected contrast image 50, and removes the background portion 61 from the map image 51 (that is, creates the background removed image 55) by performing a threshold value process.

In step S7, the image processing unit 14 superimposes the device fixed image 41 on the map image 51 so as to create the device map superimposition image 70.

In step S8, the image processing apparatus 10 (image processing unit 14) outputs an image to the display section 7 (control section 6). In this case, the device map superimposition image 70 may be output, and the display image 71 illustrated in FIG. 3 may be output. In a case where the display image 71 is output, the image processing unit 14 creates the enlarged image 44 in which the device periphery of the device emphasis image 43 is trimmed, and also displays a plurality of trimming images 45 used to apply an emphasis process to the enlarged image 44 side by side.

Thereafter, in a case where the device fixed image 41 is created for each frame, the image processing unit 14 sequentially superimposes the device fixed image 41 on the map image 51 so as to update the image. When the fluoroscopic image 40 of the latest frame is obtained, the image processing unit 14 creates the device emphasis image 43 using the fluoroscopic image 40 of the latest frame so as to update the enlarged image 44.

### Effects of First Embodiment

In the first embodiment, the following effects can be achieved.

In the first embodiment, as described above, the image processing unit 14 is configured to create the device fixed image 41 in which the device 30 introduced into the subject T is positioned such that positions thereof match each other in a plurality of consecutively generated fluoroscopic images 40, to create the map image which displays the blood vessel portion 60 in an identifiable manner with respect to the device 30 by using the contrast image 50 of a blood vessel of the subject T, stored in the storage section 12, and to create the device map superimposition image 70 by superimposing the device fixed image 41 on the map image 51. Consequently, in the map image 51, the blood vessel portion 60 can be clearly visually recognized by using the contrast image 50, and the blood vessel portion 60 can be displayed such that the device 30 is identifiable. In the device map superimposition image 70, the device 30 of the device fixed image 41 is superimposed on the blood vessel portion 60 of the map image 51, and thus the device 30 buried with a contrast agent in the typical contrast image 50 and the clear blood vessel portion 60 obtained from the contrast image 50 can be displayed together. As a result, both of the device 30 introduced into the subject T and the blood vessel portion 60 can be clearly checked in the fluoroscopic image 40 together.

In the first embodiment, as described above, the image processing unit 14 is configured to perform a process of inverting a pixel value of the blood vessel portion 60 in the contrast image 50 or a process of changing a color of the blood vessel portion 60 in the contrast image 50 to an identifiable color with respect to the device 30, so as to generate the map image 51. Consequently, it is possible to easily generate the map image 51 in which the identification of the device 30 can be improved. As a result, both of the device 30 and the blood vessel portion 60 are can be more clearly checked in the device map superimposition image 70.

In the first embodiment, as described above, the image processing unit 14 is configured to remove a part or the whole of the background portion 61 other than the blood vessel portion 60 in the contrast image 50 by using a plurality of consecutively generated contrast images 50, so as to create the map image 51. Consequently, since the background portion 61 other than the blood vessel portion 60 can be removed from the map image 51, multiplexing of the background portion 61 in the device map superimposition image 70 can be suppressed, and thus the visibility of the entire image can be improved.

In the first embodiment, as described above, the image processing unit 14 is configured to create the difference image 53 between the contrast image 50 and the past contrast image 52 of the previous frame, and to remove the background portion 61 by performing a threshold value process of removing an image component on the difference image 53 by using a threshold value of a pixel value. Consequently, it is possible to easily remove the background portion 61 while leaving the blood vessel portion 60 by using the fact that a blood vessel position changes in consecutive frames but a position of a bone or an organ which is scarcely moved does not change.

In the first embodiment, as described above, the image processing unit 14 is configured to create the device fixed image 41 on the basis of the device emphasis image 43 in which the device 30 is emphasized by using the fluoroscopic images 40 of a plurality of consecutive frames. Consequently, it is possible to improve the visibility of the device 30 in the device fixed image 41. As a result, both of the device 30 and the blood vessel portion 60 are can be more clearly checked in the device map superimposition image 70. Particularly, in cardiovascular intervention treatment, since the placed stent 31 (device 30) is emphasized, and then the blood vessel portion 60 is displayed in an identifiable manner, the degree of adhesion between the stent 31 and the blood vessel wall VW (refer to FIGS. 2B and 2C) can be easily and accurately checked, and thus it is possible to appropriately perform a determination or the like of the necessity for additional expansion using a balloon.

In the first embodiment, as described above, the device 30 includes the stent 31 for blood vessel treatment, and the fluoroscopic image 40 and the contrast image 50 are X-ray images of a part which is periodically moved due to a heartbeat of the subject T. In a case where body tissue including a blood vessel is moved periodically due to a heartbeat, such as cardiovascular intervention treatment, it is hard to sufficiently improve visibility of each of the stent 31 and the blood vessel portion 60. The image processing apparatus 10 of the present embodiment can make the blood vessel portion 60 and the device 30 identifiable by using the map image 51 while suppressing a position change by using the device fixed image 41, and is thus considerably useful in this case.

### Second Embodiment

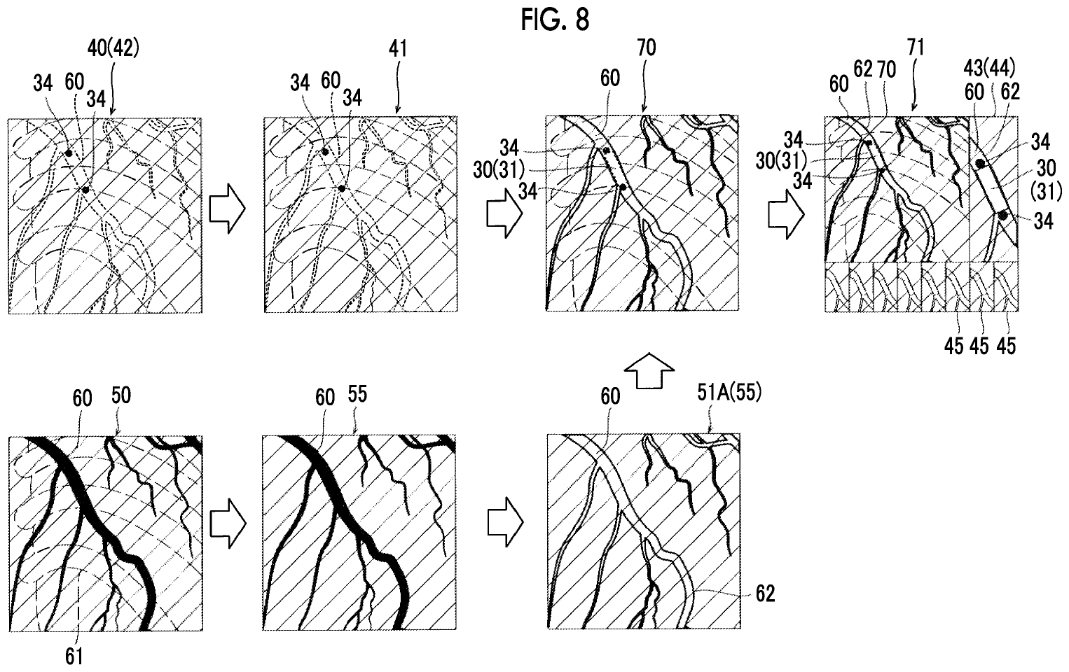

Next, with reference to FIG. 8, a second embodiment will be described. In the second embodiment, a description will be made of an example of extracting a contour of a blood vessel portion in a contrast image in addition to the first embodiment. An apparatus configuration in the second embodiment is the same as that in the first embodiment, and thus a description thereof will be omitted by using the same reference numerals.

In the second embodiment, among image processes performed by the image processing unit 14, a process (the process performed by the image processing unit 14 in step S6 in FIG. 7) regarding creation of the map image 51 is different from that in the first embodiment. In the second embodiment, as illustrated in FIG. 8, the image processing unit 14 is configured to extract a contour of the blood vessel portion 60 in the contrast image 50 so as to generate a map image (contour map image 51A) in which the contour of the blood vessel portion 60 is displayed in an identifiable manner. The contour map image 51A is an example of a “map image” in the claims.

A contour may be extracted by using a well-known edge extraction technique such as a Laplacian filter, or a method of detecting a contour (edge) on the basis of a pixel gradient. In the contrast image 50, contrast between the contrasted blood vessel portion 60 and a portion other than the blood vessel portion 60 is clarified, and thus a contour can be easily extracted with high accuracy. A contour may be extracted by using the background removed image 55 which is obtained by performing the background removal process illustrated in FIG. 5 on the contrast image 50. In this case, the unnecessary background portion 61 is removed, and thus only a contour of the blood vessel portion 60 can be extracted with higher accuracy.

As illustrated in FIG. 8, the image processing unit 14 creates the contour map image 51A in which a contour of the blood vessel portion 60 is displayed in an identifiable manner on the basis of the contrast image 50 (background removed image 55). In the contour map image 51A, a process of inverting a pixel value of the blood vessel portion 60 or a process of changing a color of the blood vessel portion 60 in the contrast image 50 to an identifiable color with respect to the device 30 is performed on a contour line 62 of the blood vessel portion 60. Consequently, in a case where the device map superimposition image 70 is created by using the contour map image 51A as the map image 51, a blood vessel wall of the blood vessel portion 60 in which the device 30 is present is displayed by the contour line 62.

In the contour map image 51A, only the contour line 62 of the blood vessel portion 60 is displayed, and the inside (the internal region of the blood vessel portion 60) of the contour line 62 may be non-colored (transparent region). In this case, in a case where the device map superimposition image 70 is created, the device fixed image 41 is displayed as it is in the internal region of the blood vessel portion 60, and thus a user (doctor) can check an actual image of the region in which the device 30 is present instead of an image created by using the contrast image 50.

The rest configuration of the second embodiment is the same as that of the first embodiment.

### Effects of Second Embodiment

In the second embodiment, in the same manner as in the first embodiment, the contour map image 51A is created by using the contrast image 50, and the device map superimposition image 70 is created by superimposing the device fixed image 41 on the contour map image 51A. Therefore, both of the device 30 introduced into the subject T and the blood vessel portion 60 can be clearly checked in the fluoroscopic image 40 together.

In the second embodiment, as described above, the image processing unit 14 is configured to generate the contour map image 51A in which a contour of the blood vessel portion 60 is displayed in an identifiable manner by extracting the contour of the blood vessel portion 60 in the contrast image 50. Consequently, in the device map superimposition image 70, only the contour (that is, a blood vessel wall) of the blood vessel portion 60 is displayed in an identifiable manner, and the device fixed image 41 can be displayed inside the blood vessel portion 60. Consequently, it is possible to generate the device map superimposition image 70 which does not give discomfort to a user familiar to the fluoroscopic image 40 unlike a case where the blood vessel portion 60 is entirely painted in an identifiable display color and in which both of the device 30 and the blood vessel portion 60 are clearly checked.

### Third Embodiment

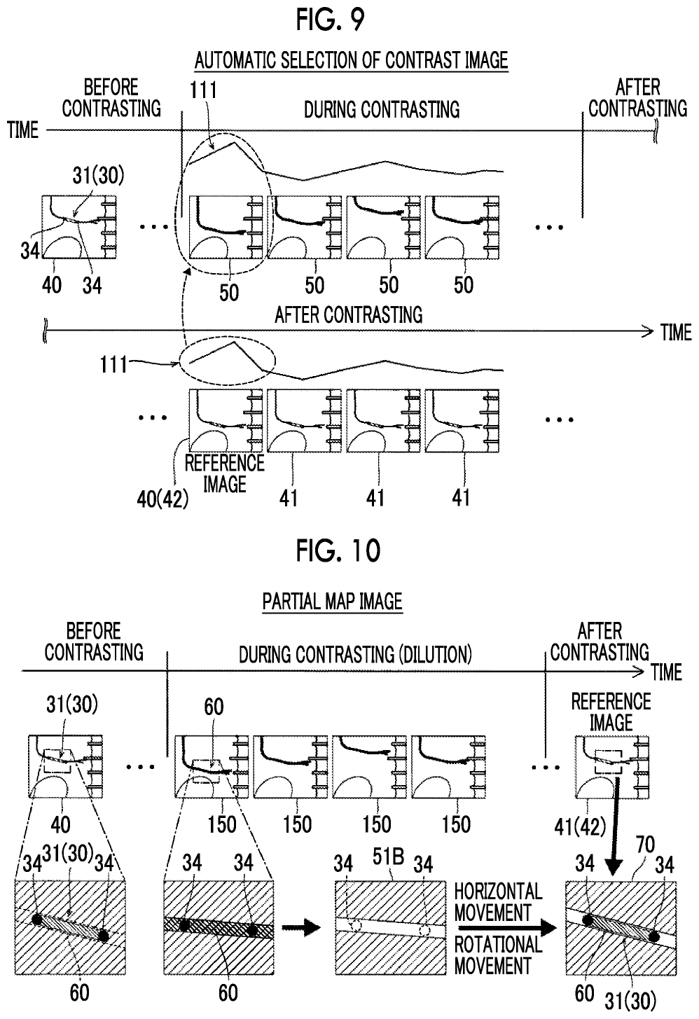

Next, with reference to FIG. 9, a third embodiment will be described. In the third embodiment, a description will be made of a configuration in which an image processing unit automatically selects a contrast image used for a map image in the first embodiment. An apparatus configuration in the third embodiment is the same as that in the first embodiment, and thus a description thereof will be omitted by using the same reference numerals.

As a method in which the image processing unit 14 automatically selects the contrast image 50 used to create the map image 51, for example, a method may be used in which the similarity between the device fixed image 41 and each contrast image 50 is calculated through image recognition. In the third embodiment, as an example of a method of selecting the contrast image 50, a description will be made of an example in which the image processing unit 14 automatically selects the contrast image 50 used to create the map image 51 on the basis of a heartbeat phase. In other words, in the third embodiment, the process in step S5 in FIG. 7 performed by the image processing unit 14 is different from that in the first embodiment.

In the third embodiment, the image processing unit may select the contrast image 50 on the basis of heartbeat phase information of the fluoroscopic image 40 (contrast image 50). A heartbeat phase indicates a timing (time position) within one cycle in a heartbeat cycle. The heartbeat phase information is information indicating a heartbeat phase at a timing of generating the fluoroscopic image 40 (contrast image 50). Since motion of the blood vessel portion 60 in the fluoroscopic image 40 and the contrast image 50 follows the periodicity of a heart stroke of the subject T, images of which heartbeat phases substantially match each other (including a case of completely matching each other) are images in which positions of the blood vessel portion 60 are similar to each other (or match each other).

The image processing unit 14 is configured to acquire heartbeat phase information of the fluoroscopic image 40 from an electrocardiographic waveform 111 or the fluoroscopic image 40, to select the contrast image 50 acquired in a heartbeat phase which substantially matches a heartbeat phase of the device fixed image 41 from among a plurality of contrast images 50 stored in the storage section 12 on the basis of the heartbeat phase information, and to create the map image 51 by using the selected contrast image 50.

As illustrated in FIG. 9, in a case where the electrocardiographic waveform 111 is used, the image processing unit 14 acquires the electrocardiographic waveform 111 in parallel with capturing of the fluoroscopic image 40 (contrast image 50), and stores the electrocardiographic waveform 111 in the storage section 12. Consequently, the image processing unit 14 can acquire a heartbeat phase (heartbeat phase information) at a generation timing of each fluoroscopic image 40 (contrast image 50) from the electrocardiographic waveform 111.

After the contrast image 50 (and the electrocardiographic waveform 111) is recorded, the image processing unit 14 determines the reference image 42, and creates the device fixed images 41 of the subsequent frames. The image processing unit 14 acquires a heartbeat phase at a generation timing of the reference image 42 used for the device fixed image 41, and selects the contrast image 50 generated in a heartbeat phase which substantially matches a heartbeat phase of the reference image 42 from among the contrast images 50 stored in the storage section 12.

The heartbeat phase information may be acquired from each fluoroscopic image 40 (contrast image 50). A method of selecting the contrast image 50 having a heartbeat phase substantially matching a heartbeat phase of the device fixed image 41 on the basis of heartbeat phase information acquired from the fluoroscopic image 40 (contrast image 50) may employ the contents disclosed in detail in Japanese Patent Application No. 2015-232474 filed by the present applicant. In the present specification, the disclosure of Japanese Patent Application No. 2015-232474 is incorporated by reference.

To summarize, the image processing unit 14 extracts a plurality of (three or more) feature points reflected in common in the device fixed images 41 (contrast images 50) through image recognition. Each feature point is a point which is periodically moved according to a heartbeat phase. The image processing unit 14 obtains a centroid position of each feature point position, and obtains a position vector of each feature point for the centroid position. The image processing unit 14 selects the contrast image 50 having a position vector group which most matches a position vector group of each feature point in the device fixed image 41, as the contrast image 50 which is generated in a substantially matching heartbeat phase. In this case, the position vector group of each feature point is heartbeat phase information. As mentioned above, the heartbeat phase information is not necessarily a heartbeat phase, and may be information indicating an image captured in heartbeat phases which match or are approximate to each other among a plurality of images.

With this configuration, the image processing unit automatically selects the contrast image 50 of which the blood vessel portion 60 matches or is approximate to the blood vessel portion 60 (refer to a dashed portion) of the device fixed image 41. The rest configuration of the third embodiment is the same as that of the first embodiment. The configuration of the third embodiment may be applied to the second embodiment.

### Effects of Third Embodiment

In the third embodiment, in the same manner as in the first embodiment, the map image 51 is created by using the selected contrast image 50, and the device map superimposition image 70 is created by superimposing the device fixed image 41 on the map image 51. Therefore, both of the device 30 introduced into the subject T and the blood vessel portion 60 can be clearly checked in the fluoroscopic image 40 together.

In the third embodiment, as described above, the image processing unit 14 is configured to acquire heartbeat phase information of the fluoroscopic image 40 from the electrocardiographic waveform 111 or the fluoroscopic image 40, to select the contrast image 50 acquired in a heartbeat phase which substantially matches a heartbeat phase of the device fixed image 41 from among a plurality of contrast images 50 stored in the storage section on the basis of the heartbeat phase information, and to create the map image 51 by using the selected contrast image 50. Consequently, in a case of the contrast image 50 during cardiovascular intervention treatment, since movement of the blood vessel portion 60 is periodic motion caused by beating of the heart, the map image 51 which accurately matches the device fixed image 41 can be selected on the basis of matching of a heartbeat phase.

### Fourth Embodiment

Next, with reference to FIG. 10, a fourth embodiment will be described. In the fourth embodiment, a description will be made of an example in which a device (markers) is also detected from a contrast image, and a local device map superimposition image in the vicinity of the device (markers) after contrasting is finished is generated, in addition to the first embodiment. An apparatus configuration in the fourth embodiment is the same as that in the first embodiment, and thus a description thereof will be omitted by using the same reference numerals.

The fourth embodiment is applied in an application form in which the degree of dilution of a contrast agent is increased compared with the contrast image 50 illustrated in FIG. 3 or 8, and thus the markers 34 of the device 30 can be detected during contrasting. Since an amount of transmitted X-rays in the blood vessel portion 60 is increased in a diluted contrast agent, suppression to pixel values at which the markers 34 can be detected is possible. In the fourth embodiment illustrated in FIG. 10, a contrast agent is used at a dilution ratio at which the markers 34 can be detected but the device 30 such as the stent 31 cannot be visually recognized. Herein, the contrast image 50 obtained by using a diluted contrast agent is referred to as a diluted contrast image 150.

As illustrated in FIG. 10, the image processing unit 14 creates a partial map image 51B in the vicinity of the device 30 in the diluted contrast image 150 generated during contrasting. Specifically, the image processing unit 14 detects the markers 34 from the diluted contrast image 150, and cuts out (trims) a region in the vicinity of the device 30 on the basis of positions of the markers 34. The image processing unit 14 performs, on the cutout image portion, a process of inverting a pixel value of the blood vessel portion 60 or a process of changing a color of the blood vessel portion 60 to a color which is identifiable with respect to the device 30, so as to generate the partial map image 51B. A contrast agent injection time is set in the storage section 12 in advance, or injection of a diluted contrast agent (a pixel value change of the blood vessel portion 60) is detected through image recognition, and thus the image processing unit 14 can automatically specify the diluted contrast image 150.

The image processing unit 14 is configured to superimpose the device fixed image 41 generated after contrasting is finished on the partial map image 51B on the basis of a device position, so as to create the device map superimposition image 70. Creation of the device fixed image 41 is the same as in the first embodiment. In a case of the fourth embodiment, since the markers 34 are also detected with respect to the partial map image 51B, one or both of parallel movement and rotational movement are performed on the partial map image 51B, and thus positioning can be performed such that positions of the markers 34 (device 30) of the partial map image 51B match positions of the markers 34 (device 30) reflected in the device fixed image 41 (reference image 42). The image processing unit 14 superimposes the device fixed image 41 and the partial map image 51B on each other by aligning the positions of the markers 34 thereof with each other.

In a case of the fourth embodiment, since the partial map image 51B can be used as a partial image of a region in the vicinity of the device 30, the diluted contrast image 150 matching or approximate to the device fixed image 41 may not be selected on the basis of a heartbeat phase or the like. This is because, in a case where the partial map image is restricted to a partial image of a region in the vicinity of the device 30, a high degree of matching can be obtained even if the diluted contrast image 150 does not match or is not approximate to the device fixed image 41.

The rest configuration of the fourth embodiment is the same as that of the first embodiment.

### Effects of Fourth Embodiment

In the fourth embodiment, in the same manner as in the first embodiment, the map image 51 (partial map image 51B) is created by using the diluted contrast image 150, and the device map superimposition image 70 is created by superimposing the device fixed image 41 on the partial map image 51B. Therefore, both of the device 30 introduced into the subject T and the blood vessel portion 60 can be clearly checked in the fluoroscopic image 40 together.

In the fourth embodiment, as described above, the image processing unit 14 is configured to create a partial map image 51B in the vicinity of the device 30 in the diluted contrast image 150 generated during contrasting, and to superimpose the device fixed image 41 generated after finishing of the contrasting on the partial map image 51B on the basis of a device position, so as to create the device map superimposition image 70. As a result, the device map superimposition image 70 can be created immediately after contrasting is finished on the basis of a device position by using a series of fluoroscopic images 40 (diluted contrast images 150) acquired during contrasting and after finishing of contrasting.

### Modification Examples

The disclosed embodiments are only examples and are not intended to be limited. The scope of the present invention is shown not by the description of the embodiments but by the claims, and includes all changes (modification examples) within the meaning and the scope equivalent to the claims.

For example, in the first to fourth embodiments, as an example, the image processing apparatus 10 used for coronary artery (cardiovascular) intervention treatment has been described, but the present invention is not limited thereto. The present invention may be applied to a radiation image processing apparatus used for applications other than the coronary artery (cardiovascular) intervention treatment. The present invention in which both of a blood vessel portion and a device can be checked together is useful to, particularly, a radiation image processing apparatus used for intravascular interventional radiology (IVR) treatment. The present invention in which a device fixed image and a map image can be superimposed on each other is useful in a case of handling a fluoroscopic image of a part where a blood vessel portion is moved among images of the heart periphery.

In the first to fourth embodiments, a description has been made of an example in which the stent 31 is used as the device 30, but the present invention is not limited thereto. In the present invention, a treatment mechanism introduced into a blood vessel may be used as a device instead of a stent.

In the embodiments, a description has been made of an example in which the present invention is applied to an image processing apparatus which performs image processing on an X-ray image using an X-ray as an example of radiation image processing, but the present invention is not limited thereto. The present invention may be applied to an image processing apparatus for a radiation image using radiation other than an X-ray.

In the embodiments, for convenience of description, a process in the image processing unit has been described by using a flow driven type flow in which processes are sequentially performed according to a process flow, but the present invention is not limited thereto. In the present invention, a process in the image processing unit may be performed according to an event driven type in which a process is performed in the event unit. In this case, a process may be performed according to only the event driven type, and may be performed according to a combination of the event driven type and the flow driven type.

What is claimed is:

1. A radiation image processing apparatus comprising:
an image generation unit that generates a fluoroscopic image based on a detection signal for radiation which is transmitted through a subject;
a storage section that stores the fluoroscopic image generated by the image generation unit; and
an image processing unit that performs image processing on the fluoroscopic image generated by the image generation unit,
wherein the image processing unit is configured to
create a device fixed image which is positioned such that positions of a device introduced into the subject match each other in a plurality of the consecutively generated fluoroscopic images,
create a map image in which a blood vessel portion is displayed in an identifiable manner with respect to the device by using a contrast image of a blood vessel of the subject, stored in the storage section, and
create a device map superimposition image by superimposing the device fixed image on the map image.

2. The radiation image processing apparatus according to claim 1,
wherein the image processing unit is configured to perform a process of inverting a pixel value of the blood vessel portion in the contrast image or a process of changing a color of the blood vessel portion in the contrast image to a color which is identifiable with respect to the device, so as to generate the map image.

3. The radiation image processing apparatus according to claim 2,
wherein the image processing unit is configured to extract a contour of the blood vessel portion in the contrast image so as to generate the map image in which the contour of the blood vessel portion is displayed in an identifiable manner.

4. The radiation image processing apparatus according to claim 1,
wherein the image processing unit is configured to remove a part or the whole of a background portion other than the blood vessel portion in the contrast image by using a plurality of the consecutively generated contrast images, so as to create the map image.

5. The radiation image processing apparatus according to claim 4,
wherein the image processing unit is configured to
create a difference image between the contrast image and a past contrast image of the previous frame of the contrast image, and
perform a threshold value process of removing an image component by using a threshold value of a pixel value on the difference image, so as to remove the background portion.

6. The radiation image processing apparatus according to claim 1,
wherein the image processing unit is configured to
acquire heartbeat phase information of the fluoroscopic image from an electrocardiographic waveform or the fluoroscopic image,
select the contrast image acquired in a heartbeat phase which substantially matches a heartbeat phase of the device fixed image from among a plurality of the contrast images stored in the storage section on the basis of the heartbeat phase information, and
create the map image by using the selected contrast image.

7. The radiation image processing apparatus according to claim 1,
wherein the image processing unit is configured to
create a partial map image in the vicinity of the device in the contrast image generated during contrasting, and
superimpose the device fixed image generated after finishing of the contrasting on the partial map image on the basis of a position of the device, so as to create the device map superimposition image.

8. The radiation image processing apparatus according to claim 1,
wherein the image processing unit is configured to create the device fixed image on the basis of a device emphasis image in which the device is emphasized by using the fluoroscopic images of a plurality of consecutive frames.

9. The radiation image processing apparatus according to claim 1,
wherein the device includes a stent for blood vessel treatment, and
wherein the fluoroscopic image and the contrast image are radiation images of a part which is periodically moved due to a heartbeat of the subject.

10. A radiation image processing method comprising:
acquiring a contrast image of a blood vessel of a subject through radiation fluoroscopic imaging;
consecutively acquiring fluoroscopic images of the subject through radiation fluoroscopic imaging;
creating a device fixed image which is positioned such that positions of a device introduced into the subject match each other in a plurality of the consecutively generated fluoroscopic images;
creating a map image in which a blood vessel portion is displayed in an identifiable manner with respect to the device by using the contrast image; and
creating a device map superimposition image by superimposing the device fixed image on the map image.
